(19) United States
(12) Patent Application Publication
(10) Pub. No.: US 2008/0146942 A1
(43) Pub. Date: Jun. 19, 2008

(54) Catheter Position Tracking Methods Using Fluoroscopy and Rotational Sensors

(75) Inventor: Praveen Dala-Krishna, Sicklerville, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MEDSYSTEMS, INC., West Berlin, NJ (US)

(21) Appl. No.: 11/610,386

(22) Filed: Dec. 13, 2006

(51) Int. Cl. A61B 5/00; A61B 8/00

(52) U.S. Cl. 600/466; 600/424

(57) ABSTRACT

Methods for determine the position and rotational orientation of the transducer array of an ultrasound imaging catheter within a patient include imaging the distal end of the catheter using fluoroscopy and determining the angular orientation based upon the shape and dimensions of the image of the transducer array and wire connecting harness. Additional rotational and translational information may be obtained from sensors located at the proximal end of the catheter. By combining position information obtained using fluoroscopy with information from relative rotation/translation sensors, the imaging transducer position and orientation can be determined more accurately. The resulting accurate imaging transducer position information enables combining multiple images from different positions or orientations to generate multi-dimensional images. Catheters including rotation and translation motion sensors at the proximal end, and radio-opaque materials near the distal end can be provided to enhance the methods.

### BACKGROUND OF THE INVENTION

1. Field of the Invention

The present invention relates generally to tracking the position of catheters used in medical procedures that are introduced into the human body, and more particularly to a method and apparatus for tracking ultrasound imaging catheters to ascertain image plane orientation and position.

2. Description of the Related Art

Ultrasound devices have been developed and refined for the diagnosis and treatment of various medical conditions. Such devices have been developed, for example, to track the magnitude and direction of motion of moving objects, and/or the position of moving objects over time. By way of example, Doppler echocardiography is one ultrasound technique used to determine motion information from the recording and measurement of Doppler data for the diagnosis and treatment of cardiac conditions, and is described in U.S. Patent Application Publication No. 20040127798 of U.S. application Ser. No. 10/620517 to Dala-Krishna, the entire contents of both of which are incorporated herein by reference.

Another ultrasound imaging technique is a class generally referred to as brightness mode (“B-Mode”) display. To generate a B-Mode display, the delay and amplitude of the received energy of ultrasound pulse echoes along different coplanar lines are measured. In B-Mode echocardiography, ultrasound energy is transmitted and subsequently reflected from the endocardial surface as well as from tissue layers within the heart. Reflected ultrasound is detected by a phased array ultrasound transducer, where the sound energy is converted into electrical pulses which can be processed to determine the direction or line from which each echo is received. The received signal amplitude along a line-of-sight is used to modulate the brightness of a line of pixels corresponding to the length of the received line, determined based upon the delay in time of the received echo, and the spatial orientation of the line. A display is thus rendered from a collection of ultrasound data where the position of each “dot” corresponds to the distance from the ultrasound transducer of a given sound reflecting object, and the brightness of each “dot” corresponds to the signal strength received from that position. A collection of coplanar lines thus forms a cross-sectional image of the subject under investigation.

The current state of the art includes ultrasonic transducers deployed in various configurations at the tips of catheters that can be introduced into the circulatory system to image various parts of the body, particularly the heart and the vascular system. Linear phased arrays, circular phased arrays, and single crystal mechanically scanning transducers are commercially available. An example of an intracardiac linear phased array ultrasound transducer is the ViewMate™ which is commercially available from EP MedSystems, Inc. of West Berlin, N.J.

The aforementioned intravascular and intracardiac ultrasound imaging techniques, combined with various imaging modalities that are possible in some of these configurations (such as Doppler and Color Doppler) have given clinicians a wide variety of tools with which to diagnose and treat various medical conditions. These tools are limited, however, in their ability to provide a comprehensive view of the underlying anatomy, and their ability to accurately track and display a plurality of moving structures and instruments, such as valves and catheters, that might be required for diagnostic or treatment purposes. 2-dimensional (2-D) images provided by known ultrasound imaging systems limit viewing to the tomographic plane currently being imaged and do not provide optimal views of structures or instruments that are not coplanar to the tomographic plane. The physician is often required to continually move the imaging catheter to “trace” a structure of interest that traverses the imaging plane. Thus, a need exists for an improved method of processing ultrasound images that allows 3-D (3-D) reconstruction of the field of view.

Although 3-D reconstruction of general ultrasonic and echocardiography images is common in the field of conventional (i.e., non-catheter) ultrasound imaging, the reconstruction of 3-D ultrasonic images using catheter based transducers has proven to be a technological challenge. Such reconstruction not only requires the ability to track and acquire images in synchrony with the cardiac activity of the subject under investigation since the heart is constantly in motion (this is termed 4-D to account for the three physical dimensions and the fourth time-dimension), but one has to accurately measure and record the relative position and orientation of the imaging plane at each viewing instant. Current catheter tracking systems common in the art, such as the use of ultrasonic ranging, use of electromagnetic fields, or body electrical impedance techniques, can be quite complicated to engineer. In particular, challenges exist in determining the rotational position of a side-firing phased array catheter with sufficient accuracy to enable reconstructing a clinically useful 3-D image.

Other methods of mechanically controlling the motion of the catheter have also been attempted. Such techniques limit the ability of the physician to manipulate the catheter while adding complexity and risk to the overall patient safety and efficacy situation. 2-D arrays capable of real-time 3-D intracardiac echocardiography have been reported in literature. However, given the severe size limitations of catheters and associated element limitations, optimal image quality has not been achieved using such techniques. Thus a need exists for a simple position tracking system that provides sufficient orientation accuracy to enable 3-D reconstruction and an associated 3-D reconstruction methodology.

### SUMMARY OF THE INVENTION

The various embodiments herein provide an apparatus and methods for tracking movement of an ultrasound imaging transducer array positioned a catheter by using fluoroscopic imaging of the imaging transducer array and associated cables. Once the position and orientation of the imaging transducer is localized using fluoroscopy, the relative orientation and position of the imaging transducer can be tracked by monitoring rotational and translational sensors on the catheter, catheter handle, sheath or insertion device (e.g., trocar).

In an embodiment method, a 2-D imaging transducer is guided into a baseline position via a catheter. The baseline position is determined and recorded using fluoroscopy. The 2-D imaging transducer captures images of the structure in interest. As the transducer is manipulated, moved and/or rotated, changes in position and orientation may be determined by noting the dimensions and aspects of fluoroscopic images of the transducer and connecting cable harness. Relative linear position and rotational orientation information may then be obtained from rotational and translational sensors disposed near the proximal end of the catheter, such as within, on or near the handle or where the catheter is inserted into a patient (e.g., introducer sheath, trocar or venal access port). As the imaging transducer is manipulated, moved and/or rotated additional images are captured and the determined linear position and rotational orientation corresponding to the image is recorded. The series of images and recorded positions can then be “stitched” together to construct a 3-D image.

In an embodiment, a rotational and/or translational position sensor uses an optical source to illuminate a part of the catheter shaft and a photosensitive sensor senses the movement of the catheter by measuring changes in the image of the illuminated portion of the catheter. In another embodiment, a magnetic rotational and/or translational position sensor uses a magnetic field sensor to sense movement of the catheter by measuring changes in magnetic field as ferromagnetic markers on the catheter pass the sensor.

Fluoroscopic position sensing offers the advantages of there being no mechanical contact between the sensor and the catheter, the ability to utilize any of the currently available phased array intracardiac catheters, and the ability to utilize existing technologies commonly found in the interventional cardiology laboratory scenario to obtain position information.

### BRIEF DESCRIPTION OF THE DRAWINGS

FIG. 1 shows an exemplary imaging system usable with various embodiments of the present invention.

FIG. 2 depicts a basic flow chart for processing 3-D images according to an embodiment of the invention.

FIG. 3 illustrates imaging planes of a phased array ultrasound imaging transducer when rotated about the long axis of a catheter.

FIGS. 4A and 4B illustrate access to the heart by cardiac catheterization via the femoral vein to the right atrium.

FIGS. 5A and 5B illustrates access to the heart by cardiac catheterization via the femoral vein to the right ventricle

FIG. 6 illustrates positioning of the ultrasound sensor in the right ventricle with catheterization via the subclavian or jugular veins.

FIG. 7 illustrates a geometrical representation of the degrees of motion that can be registered by an ultrasonic transducer deployed on a catheter.

FIGS. 8A-8I depict fluoroscopic image projections of the catheter assembly with a imaging transducer array and connecting harness in combination with an opaque insert enabling determination of the relative rotational position of the imaging transducer.

FIGS. 9A-9D depict fluoroscopic image projections of another embodiment of a catheter assembly with a crystal imaging array and connecting harness rotated about a point to determine relative rotational position of the imaging transducer an exemplary B-mode ultrasound image of the endocardium.

FIG. 10A illustrates a roller sensor for sensing rotation of the catheter within a sleeve.

FIG. 10B illustrates a magnetic rotation sensor for sensing rotation of the catheter within a sheath.

FIG. 10C illustrates an optical rotation sensor for sensing rotation of the catheter within a sheath.

FIG. 10D illustrates a roller sensor for sensing rotation of the catheter within a sheath with the sensor positioned within the catheter.

FIG. 10E illustrates a magnetic rotation sensor for sensing rotation of the catheter within a sheath with the sensor positioned within the catheter.

FIG. 10F illustrates an optical rotation sensor for sensing rotation of the catheter within a sheath with the sensor positioned within the catheter.

FIGS. 11A-11B illustrate an exemplary B-mode ultrasound image of the endocardium at diastole and systole.

FIG. 12 is a flow chart for obtaining ultrasound images and producing a 3-D composite images according to an embodiment of the invention

FIG. 13 depicts a human electrocardiogram (ECG).

FIG. 14 shows an isometric projection of the acquired image volume acquired by an ultrasound phased array imaging transducer.

### DETAILED DESCRIPTION OF THE EXEMPLARY EMBODIMENTS

Reference will now be made in detail to exemplary embodiments of the present invention. Wherever possible, the same reference numbers will be used throughout the drawings to refer to the same or like parts.

As used herein, the terms “about” or “approximately” for any numerical values or ranges indicates a suitable dimensional tolerance that allows the part or collection of components to function for its intended purpose as described herein. Also, as used herein, the terms “patient”, “host” and “subject” refer to any human or animal subject and are not intended to limit the systems or methods to human use, although use of the subject invention in a human patient represents a preferred embodiment.

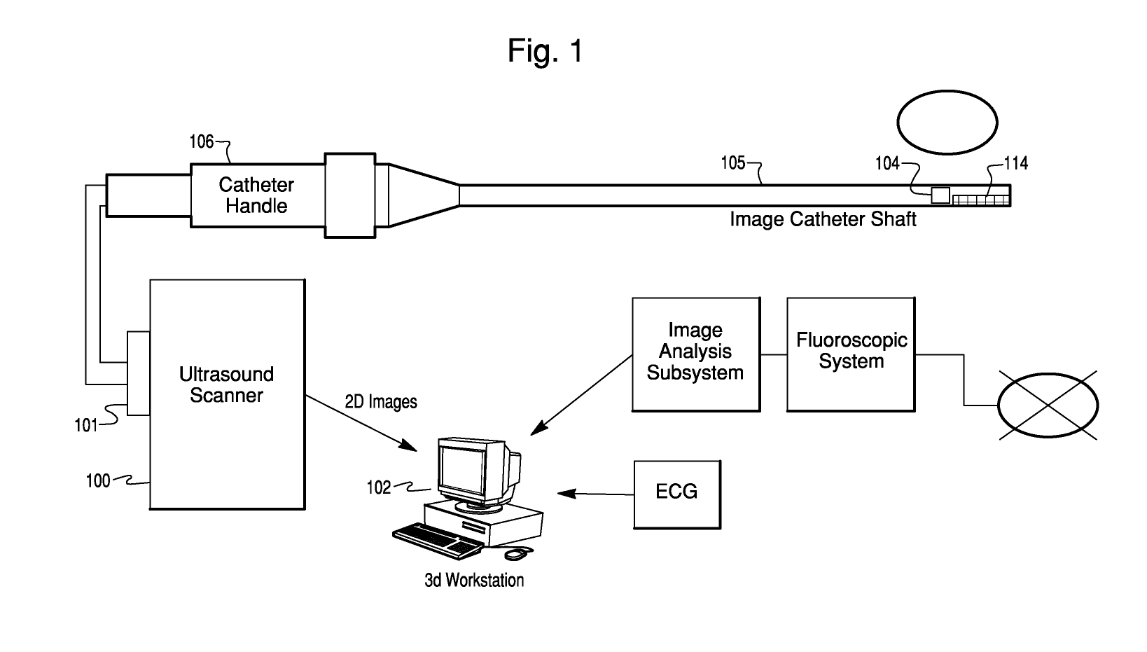

An exemplary ultrasound imaging system usable with various embodiments of the present invention is shown in the block diagram of FIG. 1. The imaging system includes an ultrasound imaging device 100, which could include within it an image processing workstation 102. The ultrasound imaging device 100 may include a display, a user interface, and an ultrasound interface all electrically coupled to a controller. The imaging system further may include a catheter handle 106 for manipulating the imaging transducer 114 disposed near the distal end of the catheter shaft 105. A connecting harness 104 can be disposed near the distal end of the catheter shaft proximate to the crystal array of the imaging transducer 114.

The system may further include an electrical interface 101 between the catheter handle 106 and the ultrasound scanner 100 for electrically isolating the catheter from the rest of the system to protect the patient from induced and fault currents. The electrical interface 101 can include isolation circuits on all conductors coupled to the catheter as disclosed in U.S. patent application Ser. No. 10/997,898, which published as U.S. Publication No. 2005-0124898, the entire contents of which are incorporated herein by reference. Also, the electrical interface 101 may include tissue temperature sensing and protection circuits, such as disclosed in U.S. patent Ser. No. 10/989,039, which published as U.S. Publication No. 2005-0124899, the entire contents of which are incorporated herein by reference.

The various parts that form the ultrasound system illustrated in FIG. 1 can be within the same unit, physically or functionally integrated at various levels, or one or more of these could be separately deployed as one or more separate units with various means of inter-communication of signals and control.

Various embodiments utilize a 2-D imaging transducer 114 mounted on a catheter delivery system to generate a series of 2-D images in coordination with positional information which can be used to generate 3D image constructions. Additional information regarding rotational position or orientation of the catheter, particularly about the long axis of the catheter, can enable generation of more accurate 3-D images.

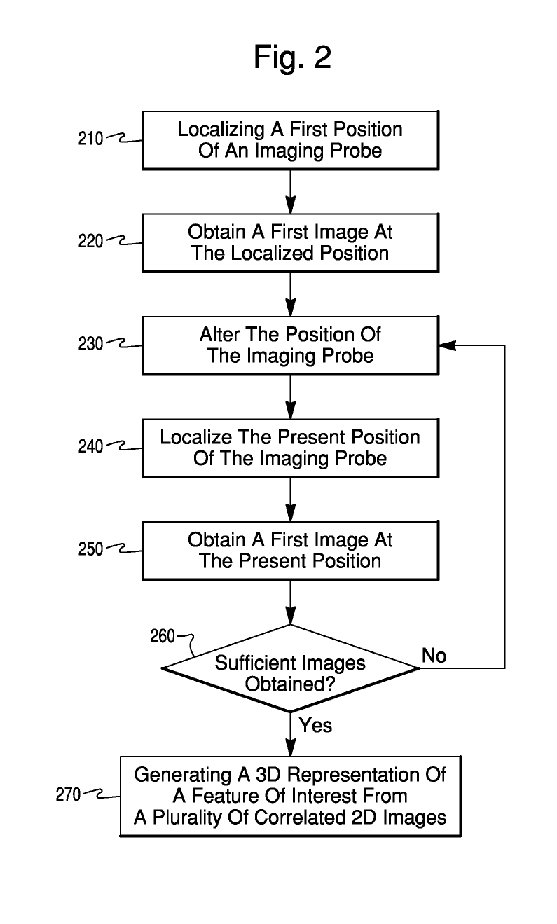

According to an embodiment as shown in FIG. 2, the system may be used to generate a still or moving 3-D representation of a feature of interest from a plurality of correlated 2-D images. Referring to FIG. 2, in step 210, sensors disposed on the imaging catheter (e.g., an intra-body ultrasound catheter probe forming part of ultrasound equipment 110) can be used to localize a position of an imaging probe (e.g., an ultrasound phased array transducer). Such a process is described in U.S. patent application Ser. No. 10/994,424 entitled “Method And Apparatus For Localizing An Ultrasound Catheter”, the entire contents of which are incorporated by reference herein.

In one exemplary embodiment for performing step 210 (and step 240), fluoroscopy techniques disclosed herein are utilized to determine the linear and rotational position. This embodiment may be used to position the imaging transducer 106 at a baseline position near the anatomical structure of interest. This baseline position is then recorded, such as by recording the fluoroscopic image of the distal portion of the catheter.

Once the baseline position and orientation of the ultrasound imager has been localized in step 210, a first series of time-gated images is obtained in step 220 at the localized position according to various embodiments. This series of images can be stored along with the localizing information for subsequent 3-D generation to be described in detail below.

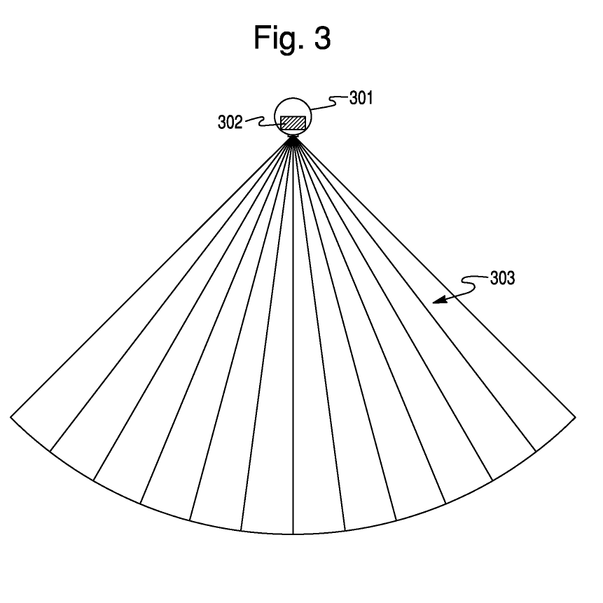

Once the series of images of the baseline position and/or rotation of the transducer array have been obtained, the position and/or rotation of the imaging catheter is altered in step 230, such as by a user rotating the intra-body catheter so that the transducer array thereon is facing a new direction. At each rotational position or step, a 2-D image can be obtained by the transducer 302 along a thin slice or plane 303, referred to herein as an “imaging plane,” as illustrated in FIG. 3. The step size or rotation angle can be selected so images cover adjoining slices or volumes (perhaps with some overlap) so that a 3-D image(s) can be reconstructed by “stitching” the 2-D slice images together, such as in the workstation 100. In another embodiment, orientation information obtained from fluoroscopic images can be provided to the user to assist in rotating the transducer array to a proper orientation for obtaining a next imaging plane. In a further embodiment, the timing of each image slice can be controlled at least partly by use of a physiological trigger, such as a movement of a structure or an electrocardiogram (ECG) signal event. Such a physiological trigger can be matched beforehand or by user selection with a specific portion of a structure for imaging.

Fluoroscopy techniques image the connecting harness 104 within the catheter and imaging transducer 106 to obtain image data that can be used to localize the present (second) position and orientation (i.e., after alteration in step 230) of the imaging transducer 106. In step 250, a subsequent image or series of ultrasound images is obtained at the present location, and these images are stored along with the timing/triggering and localizing information as previously described. Steps 230, 240 and 250 can be repeated until images from a sufficient number of imaging perspectives have been obtained for 3-D rendering (see step 260). According to an embodiment, steps 230, 240 and 250 are continued even after a 3-D representation has been generated in step 270 in order to update or refresh the 3-D representation with the latest images.

A 3-D representation of a feature of interest may then be generated in step 270 by workstation 100 from the plurality of 2-D images obtained in steps 220 and 250. The ultrasonic signals produced at each position can be correlated, stitched together or otherwise matched up in accordance with known techniques.

Thus, a first step involves localizing the imaging transducer at a baseline position near the structure of interest. Although the method and apparatus of the invention can be used to generate 2-D and 3-D images of virtually any anatomical structure, the invention is described herein with reference to embodiments used to generate 2-D and 3-D images of the endocardium and its sub-structures. Accordingly, exemplary reference will be made to the endocardium and its sub-structures. To acquire images of the endocardial surface of the right and left ventricle, a phased array ultrasound imaging catheter is positioned within the heart via percutaneous cannulation using standard cardiac catheterization techniques of the femoral vein or the subclavian or jugular veins.

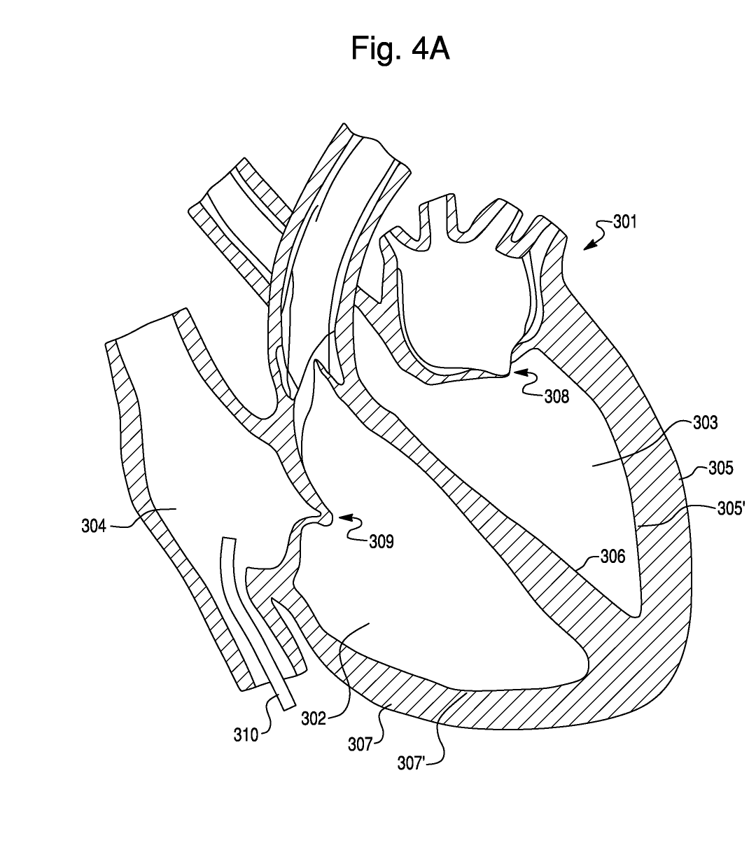

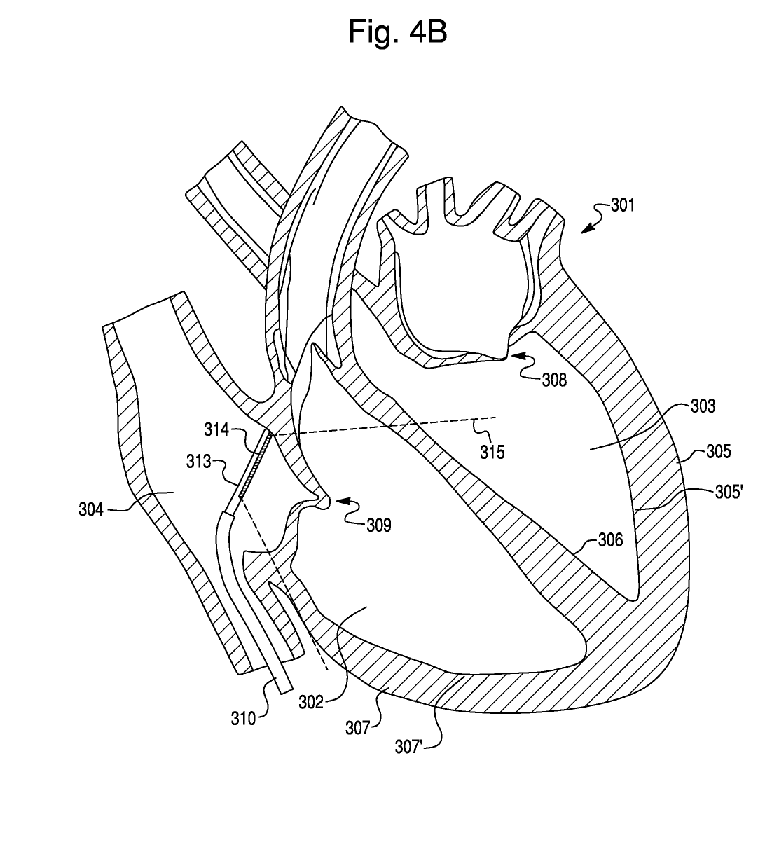

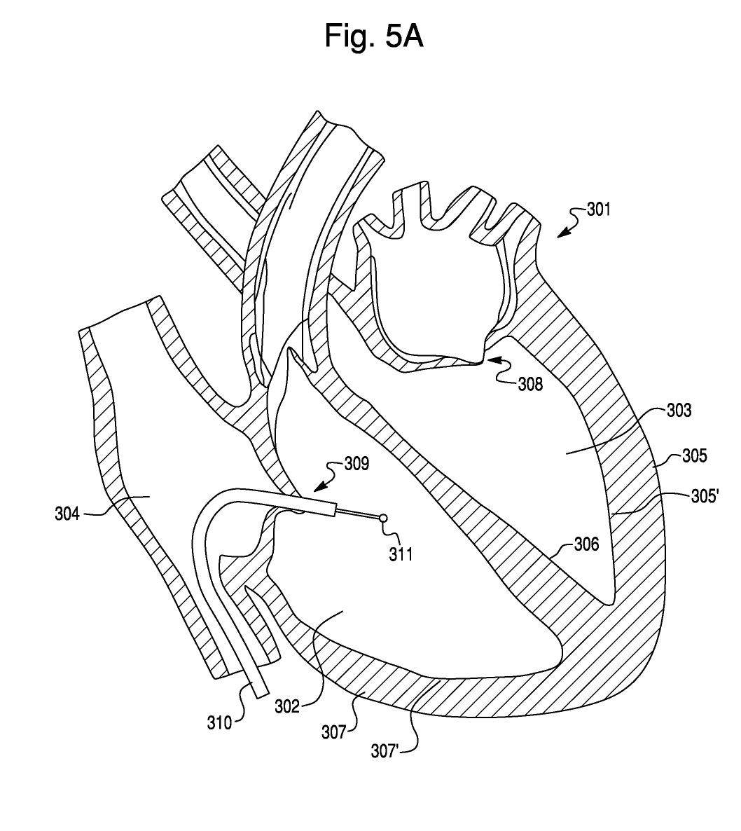

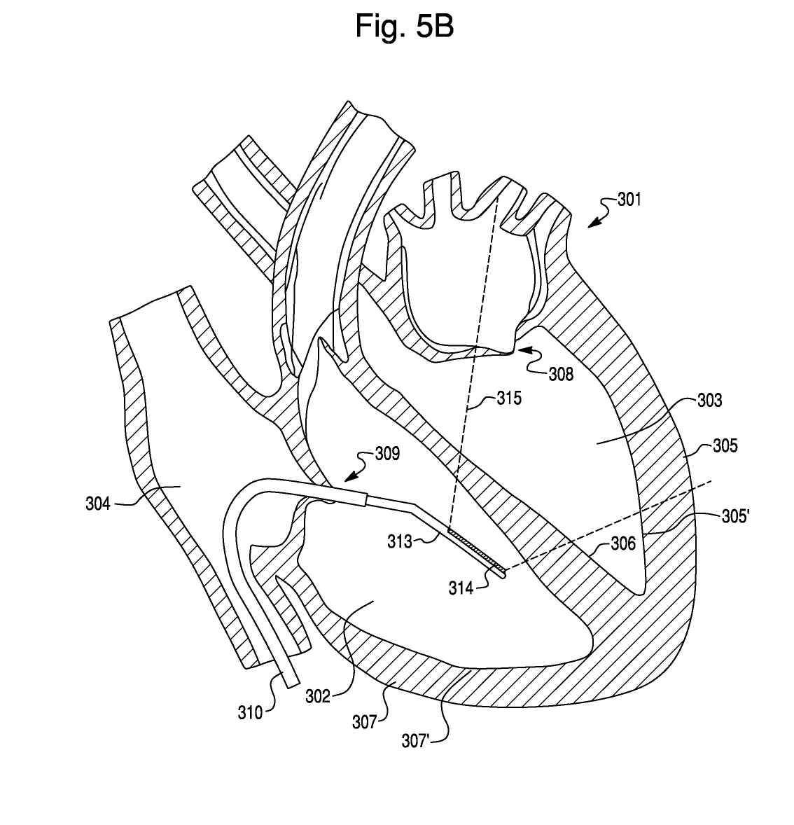

In order to properly position the phased array ultrasound imaging catheter, a long preformed intravascular sheath 310 can be advanced under fluoroscopic control into the various chambers of the heart 301, as shown in FIG. 4A. The sheath may be sufficiently transparent to ultrasound or has an ultrasonic deployable window. FIG. 4A illustrates access to the heart by cardiac catheterization via the femoral vein to the right atrium 304. A guide wire 311 (shown in FIG. 5A) may be used to properly position the sheath in or near the orifice of the tricuspid valve. Once the sheath is positioned with its distal end in the right atrium 304, the phased array ultrasound imaging catheter 313 can be advanced through the sheath until the ultrasound transducer 314 is properly positioned outside the tricuspid valve 309 for imaging the right ventricle 302, as shown in FIG. 4B. In this position, the field of view 315 (indicated by dotted lines) of the ultrasound transducer 314 can address most, if not all of the right ventricle 302, right ventricle wall 307 and much of the septum 306.

For imaging the left ventricle 303, the ultrasound transducer 314 needs to be positioned within the right ventricle. This can be accomplished by passing a guide wire 311 through the sheath 310, and under fluoroscopy control, passing the guide wire 311 through the tricuspid valve 309. The sheath 310 is then directed over the guide wire 311 into the orifice of the tricuspid valve 309 and advanced into the right ventricular cavity 302, as illustrated in FIG. 5A. Once the sheath 310 is properly positioned, the guide wire 311 is withdrawn and the phased array ultrasound imaging catheter 313 is advanced under fluoroscopic control through the sheath 310 into a position inside the right ventricular in the mid cavity region, as illustrated in FIG. 5B. In this position, the field of view 315 (indicated by dotted lines) of the ultrasound transducer 314 can address most, if not all of the left ventricle wall 305 and much of the septum 306.

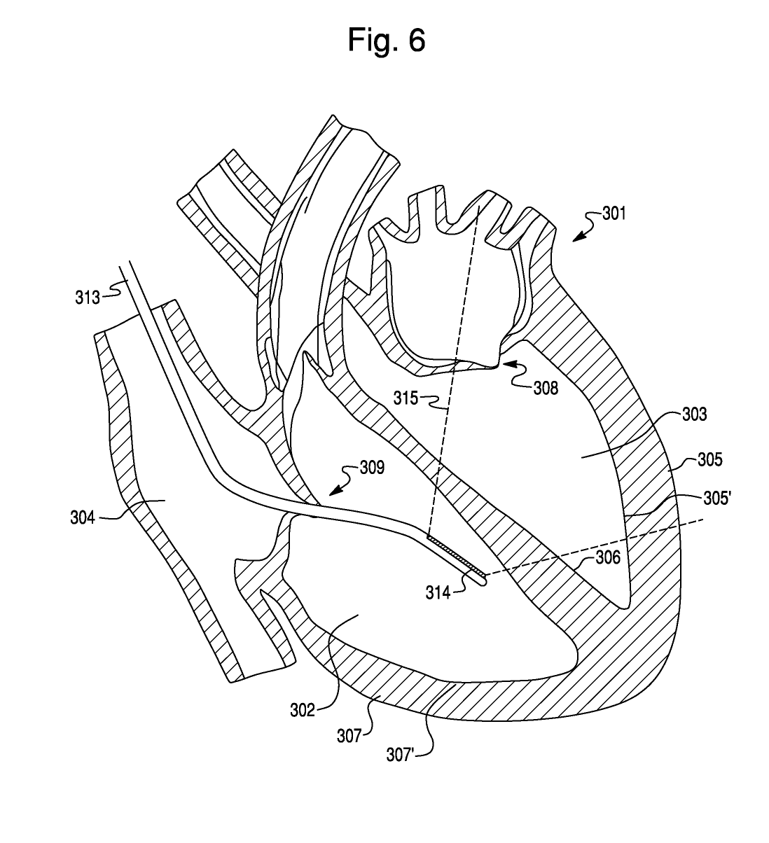

Other methods other than the use of a sheath 310 to position the ultrasound phased array catheter 313 in the heart 1 can be employed. For example, a steerable ultrasound catheter, such as disclosed in U.S. Patent Publication 2005/0228290, can be used and guided directly under fluoroscopy control into position within orifice of the tricuspid valve or within the right atrium, as illustrated in FIG. 6. FIG. 6 illustrates positioning of the ultrasound sensor 314 in the right ventricle 302 with catheterization via the subclavian or jugular veins.

In order to be able to correlate and stitch together multiple 2-D images to render a 3-D reconstruction, the position of the imaging probe, such as an ultrasound phased array transducer, at the instant a 2-D image is obtained needs to be accurately. Various methods for localizing a catheter within the body are disclosed in U.S. application Ser. No. 10/994,424 incorporated by reference above. However, in a moving organ such as the heart, forces from flexing muscles and flowing blood can cause the catheter to move about rapidly. Such movements may have a shorter interval than the positioning resolving time of known positioning methods. For this reason, additional positional information may need to be used to narrow the position error on 2D images sufficient to render clear 3D images. Fluoroscopic images capture the position and orientation of the imaging transducer 114 at an instant in time, and thus have near-instantaneous resolving time which reduces position and orientation measurement errors caused by heart movements. In some embodiments additional fast responding position or movement sensors may be added to the catheter to supplement or update imaging probe position information obtained using fluoroscopic methods described herein.

The orientation of the transducer array about the catheter's longitudinal axis is of particular importance when 2-D image slices are assembled into a 3-D image. As shown in FIG. 3, a transducer 303 in one position can scan several slices 303 based upon its angular orientation. Similarly, the orientation of the transducer array about its midpoint is important to determine the viewing perspective from a particular point within the patient.

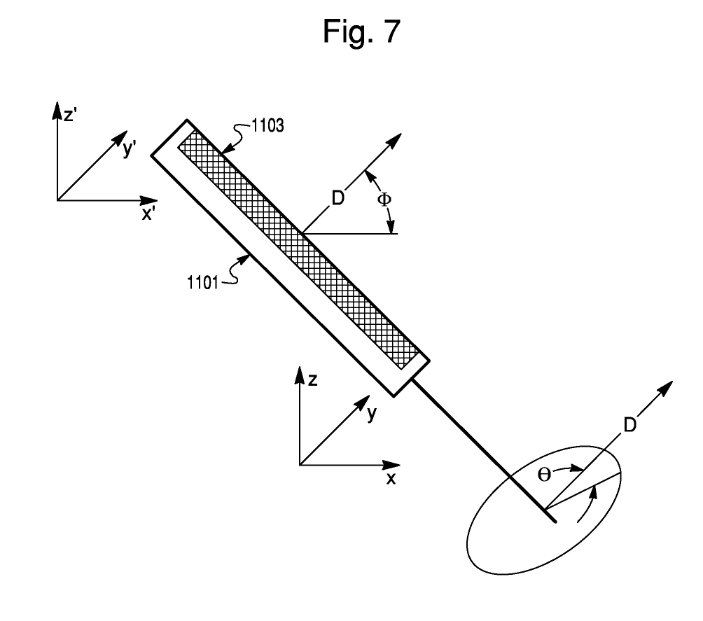

The importance of determining the two rotational orientations in order to assemble a 3-D image can be appreciated by referring to FIG. 7 which shows a geometrical representation of the degrees of freedom (position and orientation) of an ultrasonic transducer 1103 deployed on a catheter 1101. A side-firing phased array 1103 is shown in FIG. 7 with the catheter deployed at the tip of the imaging catheter 1101. Although a side-firing phased array transducer is shown in FIG. 7 with the catheter deployed at the tip of the catheter, other imaging formats and transducers, including circular arrays, mechanically scanned transducers and other transducers can benefit from this invention. Further, transducers can also be deployed in the body of the catheter and not necessarily at the tip.

In FIG. 7, the direction D represents the mid-line of the 2D imaging plane (shown in the plane of the image) which extends along a plane parallel to the length of the array and perpendicular to the face of the sound emitting faces of the array elements. As shown, the transducer tip is capable of being located in 3 dimensional space (represented by x′, y′, z′). The base of the transducer can also be located in space through x, y, z dimensions. Further, the transducer is capable of being rotated through an angle of θ, around its longitudinal axis. More broadly, the transducer array may move through six degrees of freedom that may need to be accounted for in constructing and combining images. Specifically, the array may be positioned in 3-dimensional space of left/right, up/down and in/out (with respect to the long axis of the catheter), rotated through angle θ (roll angle) and oriented so the linear array is tilted up/down (inclination or pitch angle) and angled left/right (yaw angle).

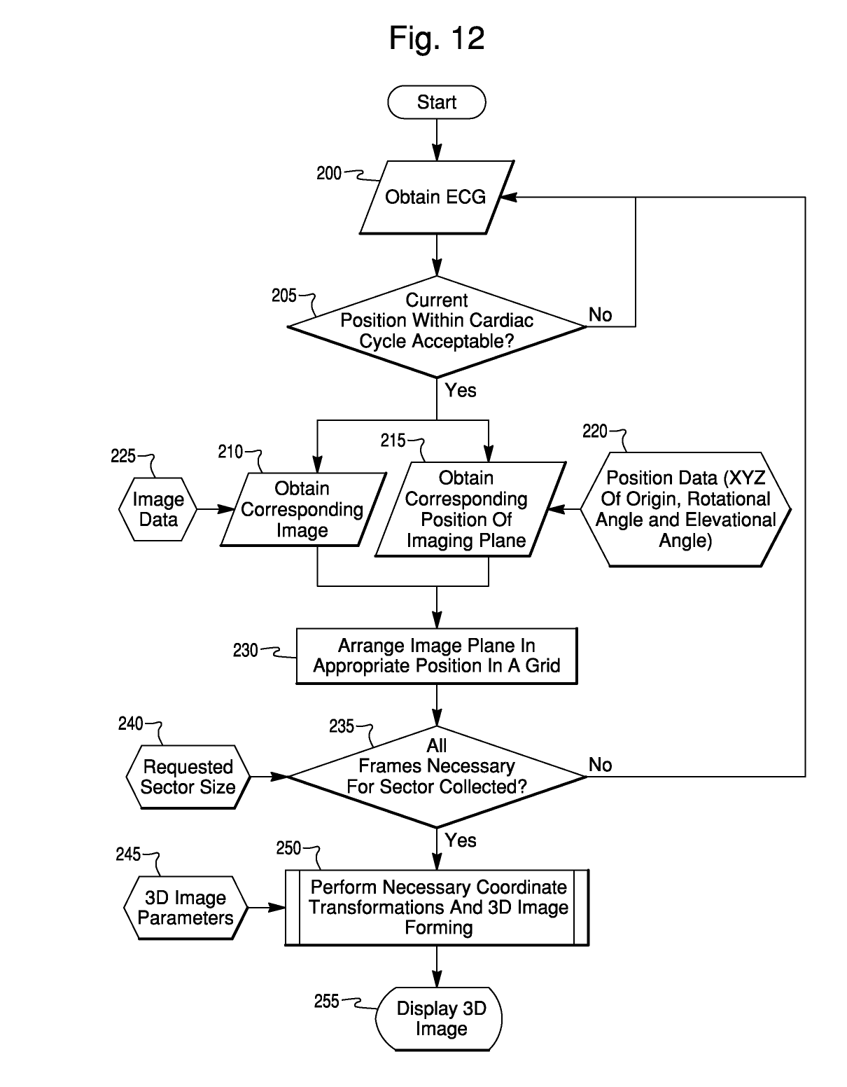

In another representation, the origin of the imaging midline (D), can be located in space (e.g., x, y, z). The inclination, yaw and rotation of the imaging plane can then be measured against any arbitrarily assigned axis as rotation θ, and inclination φ, as well as yaw angle (not shown in FIG. 7 as this motion is with respect to the plane of the image). From either of the above three coordinate definitions of position, the relative positions of the imaging planes, can be easily identified as the transducer is rotated and moved through space. This positional information then can be utilized in image processing, such as shown in the embodiment illustrated in the flow chart in FIGS. 2 and 12.

The fluoroscopic technique to determine linear and rotational position via the projected image of the crystal imaging array and connecting harness described above provide information necessary for the processor to solve the geometric relationships in order to locate the transducer in 3-D space. Linear fluoroscopic readings of the catheter which sense the axial deployment of the catheter within the sheath can provide X, Y, Z axis position information with respect to the catheter. The analysis of the projected image of the crystal imaging array and connecting harness can provide the rotation angle θ or inclination angle φ data with respect to the catheter.

In various embodiments of the ultrasound imaging catheter, an ultrasound pulse/echo signal cable connecting harness 104 is present within the catheter in the vicinity of the imaging transducer array 114. The connecting harness 104 includes many (e.g., 64) coaxial cables that carry ultrasound electrical pulses from an ultrasound system to the piezoelectric crystal transducers within the imaging transducer array 114 and return ultrasound echo signals from the transducers to the ultrasound system. Comprising many parallel electrical conducting wires packed closely together, the connecting harness 104 presents a strip of radio opaque material within the catheter which registers a projected image when a fluoroscopic scan, such as X-ray, is taken. As X-rays pass through the patient, they are attenuated by varying amounts as they interact with the different internal structures of the body, casting a shadow on the fluorescent screen of X-ray absorbing structures. When the ultrasound imaging catheter with connecting harness 104 and imaging transducer 114 are positioned within the X-ray beam, both the imaging transducer array and connecting harness also “cast a shadow” on the fluoroscopic image. By noting the configuration and linear position of the shadowed image of the connecting harness 104 and imaging transducer 114, the linear position and orientation of the imaging transducer 114 can be determined.

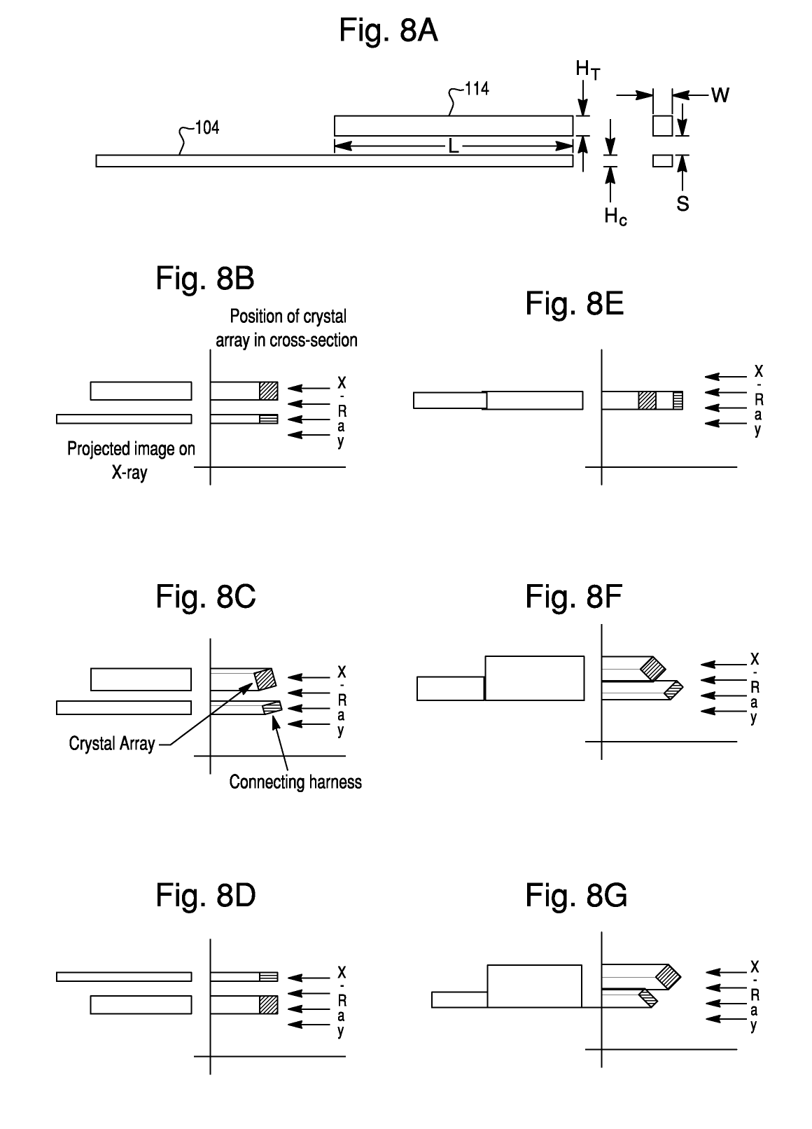

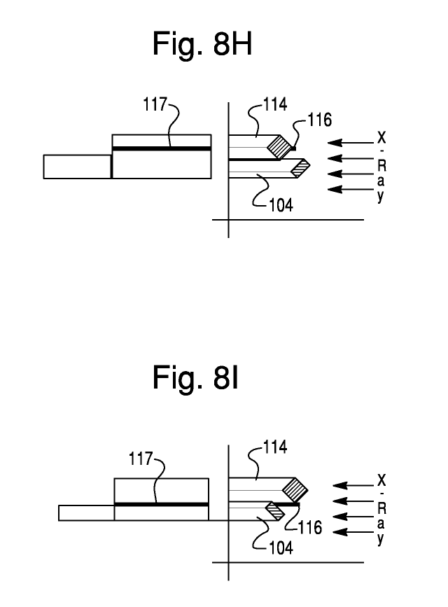

FIGS. 8A-8G depict the resulting image when x-rays are impinged on the transducer imaging array 114 and connecting harness 104 at various angles. The position of the transducer imaging array 114 and the attached connecting harness 104 are seen as two separate lines on the fluoroscopic system, with the thicker line corresponding to the imaging transducer array 114 and the thinner line corresponding to the connecting harness 104. The views in FIGS. 8B-8G show that as the rotational orientation of the transducer imaging array with respect to the X-ray imaging plane changes, the shape and orientation of the two shadows vary in predictable ways. In these figures, the orientation of the transducer array is illustrated on the right side of the vertical line while the resulting X-ray image is illustrated on the left side.

FIG. 8A illustrates the dimensional characteristics of the imaging transducer and connecting harness that need to be known in order to determine the orientation of the transducer array. Such dimensions include the length L of the transducer array, the transducer height H_{T}, the connecting harness height H_{C}, the width W of the transducers and connecting harness, and the separation S between the transducers and connecting harness. For example, the yaw angle (i.e., rotational orientation into and out of the plane of FIG. 8A) can be determined by comparing the length of the transducer array in the image to L using simple trigonometry. The ambiguity in the yaw angle inherent in this measure (i.e., the angle could be either into or out of the page) can be resolved by taking another fluoroscopic image at a different viewing angle.

The orientation of the transducer array about the longitudinal axis can be determined by comparing the measured heights, width and separation in the image to the known dimensions of the imaging catheter. For example, FIG. 8B depicts the projected X-ray image when the X-ray scan is orthogonal to the transducer imaging array 114 and the attached connecting harness 104 (i.e., the imaging plane of the transducer is parallel to the X-ray image plane and oriented toward the top of the figure). As shown the X-rays through the transducer imaging array 114 casts a larger X-ray shadow than the connecting harness 104 which is imaged edge on in this orientation. In this imaging configuration, the height of the transducer array and the connecting harness measured in the X-ray image will match H_{T} and H_{C}, respectively, while these two images will be separated by the distance S.

As the catheter with both imaging transducer array and connecting harness are rotated about the longitudinal axis, the relative width and position of the resulting projected x-ray images vary. As shown in FIG. 8C, where the transducer array imaging plane is tipped outward from the plane of the figure, the measured heights of the transducer array and connecting harness on the X-ray image will exceed the known values H_{T} and H_{C}, respectively. Additionally, the separation between the two images will be less than the value S. The angle of orientation with respect to the plane of the X-ray image can be determined based upon simple trigonometry.

FIG. 8D depicts the projected X-ray image when the X-ray scan is orthogonal to the transducer imaging array 114 and the attached connecting harness 104 but the imaging plane is oriented toward the bottom of the figure. In this imaging configuration, the height of the transducer array and the connecting harness measured in the X-ray image will match H_{T} and H_{C}, respectively, these two images will be separated by the distance S, and the connecting harness image appears above the transducer array image.

FIG. 8E depicts the projected X-ray image when the X-ray scan is parallel to the transducer imaging array 114 and the attached connecting harness 104, i.e., the X-ray imaging plane is orthogonal to the ultrasound imaging slice. In this imaging configuration, the width of the transducer array and the connecting harness measured in the X-ray image will match W, however these two structures will be superimposed and thus not separately resolvable. If the width of the connecting harness is greater or less than the transducer array, then the overlap of these two structures may be seen in the image as a change in shadow thickness. This imaging configuration may introduce ambiguity as to whether the ultrasound imaging plane extends into or out from the X-ray imaging plane. Such ambiguity may be resolved by obtaining an X-ray image from a different viewing perspective or by one of the methods described further below.

As the catheter with both imaging transducer array and connecting harness are rotated further about the longitudinal axis, the separation between the transducer array and connecting harness images will diminish and, with sufficient rotation, disappear. FIG. 8F shows a configuration where the transducer array imaging plane is tipped sufficient outward from the plane of the figure that the shadows of the transducer array and connecting harness overlap. In this configuration, the width of the shadow of the catheter distal end equals the diagonal dimension of the transducer array and connecting harness structures. These structures can then be resolved at the proximal end of the transducer array.

Thus, the rotational position of the transducer can be calculated by the relative thicknesses and extent of overlap of the shadows from the crystals and the harness.

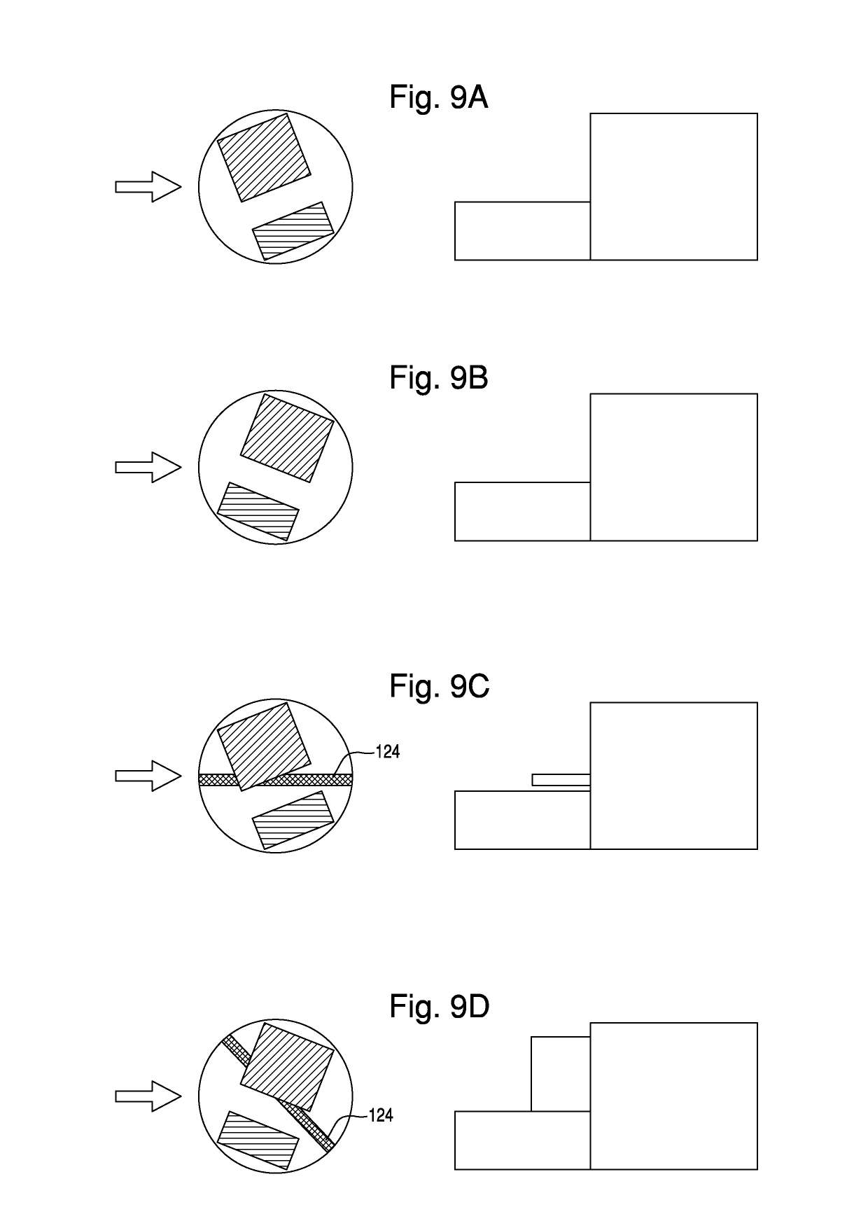

As illustrated in FIG. 8G compared to FIG. 8F, and FIG. 9A compared to 9B, ambiguity in the orientation of the transducer array may arise regarding whether the angle of rotation with respect to the fluoroscopic imaging plane is into or out of the plane. Such ambiguity may arise because the relative position of or overlap between the transducer array and connecting harness shadows may be similar at equal angles or rotation into or out of the fluoroscopic imaging plane (i.e., at equal angles to the transducer imaging plane). This ambiguity is due to the bilateral symmetry (approximate) about the ultrasound imaging plane of the transducer array and connecting harness illustrated in the figures. Thus, for a transducer with bilateral symmetry, the ultrasound imaging plane defines an axis of symmetry of the transducer array in 2-D X-ray images, such that while the magnitude of the angle of the ultrasound imaging plane with respect to the X-ray image can be determined, the direction of the imagine plane into or out of a single X-ray image plane can not be resolved. As mentioned above, such ambiguities can be resolved by fluoroscopic imaging from different perspectives, i.e., two X-ray image planes.

Another solution to the ambiguity problem involves adding a radio-opaque material (i.e., a material that absorbs X-rays) to the catheter in the vicinity of the transducer array with a shape, orientation and location that provides additional orientation information in the X-ray image. Examples of radio-opaque materials include metals such as aluminum, barium, nickel, zinc, tungsten, and copper, non-metals, such as iodine, and compounds, such as sodium chloride, calcium iodide. Radio-opaque materials used for this application may be selected to maximize X-ray absorption in the wavelengths (which are related to the X-ray energy) used in cardiac fluoroscopy. Such radio-opaque materials may be positioned asymmetrically (e.g., near the exterior of the catheter body along a radian at an acute angle to the imaging plane of the transducer array), in a plane at an acute angle to the imaging plane of the transducer array, or in other orientations and configurations that will result in different X-ray images when the ultrasound imaging plane is at an angle into the X-ray image plane than when the ultrasound imaging plane is at an angle out of the X-ray imaging plane. Examples of this embodiment are illustrated in FIGS. 8H and 8I, and 9C and 9D.

Referring to FIGS. 8H and 8I, a wire, small diameter rod or thin section of radio-opaque material can be positioned in an asymmetrical location within the catheter at position with a radian (i.e., the line through the material and the centerline of the catheter) at an acute angle to the imaging plane of the transducer array. Being located on a radian at an acute angle to the imaging plane means the radio-opaque has a different axis of symmetry about the catheter longitudinal axis for 2-D X-ray images. In other words, the angle ambiguity (i.e., the angle of rotation that may be either into or out of the plane of the image) of the radio-opaque material is different from the angle ambiguity of the transducer array. For example, the radio-opaque material can be positioned near the outside edge of the catheter or on a corner of the transducer array as shown in FIGS. 8H and 8I. The radio-opaque material will cast a dark shadow (due to the greater absorption of X-rays through the material than through the transducer array) that can be located on the X-ray image. Due to its asymmetrical position and different axis of symmetry, the shadow of the radio-opaque material appears in a different position on the images when the transducer array imaging plane is oriented into or out of the X-ray imaging plane, thereby removing angular ambiguity from the image.

A slightly different implementation of this embodiment is illustrated in FIGS. 9A-9D. As illustrated in FIGS. 9A and 9B, a transducer array and connecting harness may cast the same shadow when the transducer imaging plane is tilted the same number of degrees away from the X-ray imaging axis and toward the X-ray imaging axis. This ambiguity can be removed by providing a thin planer piece of radio-opaque material in the catheter near the imaging transducer array oriented at an acute angle to the transducer array imaging plane. An example of this embodiment is illustrated in FIGS. 9C and 9D, where the radio-opaque sheet 124 is positioned on the proximal end of the transducer array and oriented at an acute angle to the transducer imaging plane. When the catheter is oriented so the transducer imaging plane tilts toward the X-ray source as shown in FIG. 9C, the radio-opaque sheet 124 is positioned to present a narrow profile to the X-rays, leaving a thin shadow on the X-ray image. In contrast, when the catheter is oriented so the transducer imaging plane tilts away the X-ray source as shown in FIG. 9D, the radio-opaque sheet 124 is positioned to present a wide profile to the X-rays, leaving a large shadow on the X-ray image. This difference in the X-ray images between 9C and 9D reveals how the angular ambiguity illustrated in FIGS. 9A and 9B can be resolved using this method.

Embedded directional markers within the body of the catheter transducer are also envisaged, which can contribute by either improving the accuracy of the estimated position, or by enabling the use of lower quality or lower dose fluoroscopic systems. Such markers can include single or multi-planar shapes made of radio-opaque material that vary the characteristics of the fluoroscopic shadows cast by the differing geometrical profile or X-Ray attenuation as a function of rotation.

Radio-opaque markers may also be placed on the chest and/or back of a patient in areas that will not impede the procedure to allow the X-ray positioning methods to compensate for physical movements of the patient. Such markers will be recorded on the same X-ray image as those used to determine the position and orientation of the imaging transducer array. Such markers may then be used as fiducial reference points for locating the catheter with respect to the body. Such markers on the chest (assuming the patient is lying on his/her back) will move with the chest, and thus indicate movements of the chest cavity, such as due to breathing.

When using fluoroscopic methods for determine the position and rotational orientation of the imaging transducer array, image subtraction methods may be employed to enhance the rotational information provided in the fluoroscopic images. Such methods can essentially remove the portions of the image that are unchanged, revealing only those portions that changed from one image to the next. Such methods, thus, can reveal the change in the transducer array/connecting harness shadow, and thus the change in the rotational orientation. Such a method may be particularly useful when a series of ultrasound images are to be obtained and the catheter is rotated between each ultrasound image.

Information from a fluoroscopic imager can be digitized and provided to a processor, such as the ultrasound system processor, which can be programmed to determine the position and orientation of the transducer array according to the methods described above. The processor may be programmed to recognize and determine the dimensions of the image pattern of the transducer array and the connecting harness. Then using simple trigonometric algorithms or a simple look-up table, the processor can calculate the orientation associated with the image pattern. Such a processor may estimate the catheter position/orientation in real time, or store the fluoroscopic image information along with ultrasound image information for later processing. Also, the processor may be programmed to update position and orientation information obtained from fluoroscopic methods.

In another embodiment, rotation and/or translational sensors are provided on the proximal end of the catheter, such as in or near the handle, to provide rotational information. By sensing aspects on the catheter itself, such sensors can provide a near instantaneous measure of rotational and/or translational (i.e., in or outward) movement of the catheter with respect to the opening into the patient. By combining information obtained by fluoroscopic imaging methods as described above with near-instantaneous catheter rotation/translation movement information, a more accurate measure of the imaging transducer position and orientation may be obtained, particularly during intervals between fluoroscopic images. Thus, providing such sensors on the catheter can be used to enhance the fluoroscopic methods described above to reduce positional/rotational orientation errors, reduce X-ray exposure (e.g., by reducing the necessary X-ray frame rate), or accommodate continuous rotational/translational catheter motion.

While the position of the distal end of the catheter can only be inferred from the relative motion measure at proximal end, when such information is combined with other position measuring methods, such as the fluoroscopic methods described above, an accurate estimation can be obtained. For example, accurate measures of transducer array orientation at a few different angles of rotation, such as measured using fluoroscopy, can be compared to rotational movement sensor information to effectively calibrate the rotational movement sensor to actual rotational movements.

Rotational and/or translational sensors may be provided at the proximal end of the catheter in order to measure movement with respect to the handle, a sheath surrounding the catheter, an introducer (e.g., a trocar or venal access port), or the body of the patient. A catheter is positioned within a body by pushing it into or pulling out from an opening, such as an access port that has been inserted into the femoral vein to provide access to the right ventricle. The term “venal access port” is used herein to refer to a device inserted into a vein or artery in order to provide access for inserting a catheter into the vessel. The orientation of the distal end of the catheter may then be adjusted by rotating the catheter clockwise or counterclockwise by turning the proximal end. Thus, measurements of relative motions at the proximal end can be related to changes in position of the distal end. Since the proximal end exits the body at a fixed point, catheter movement sensors can be located outside the body. This allows use of sensors that cannot be made small enough to fit within the size limitations on the distal end of an intracardiac catheter.

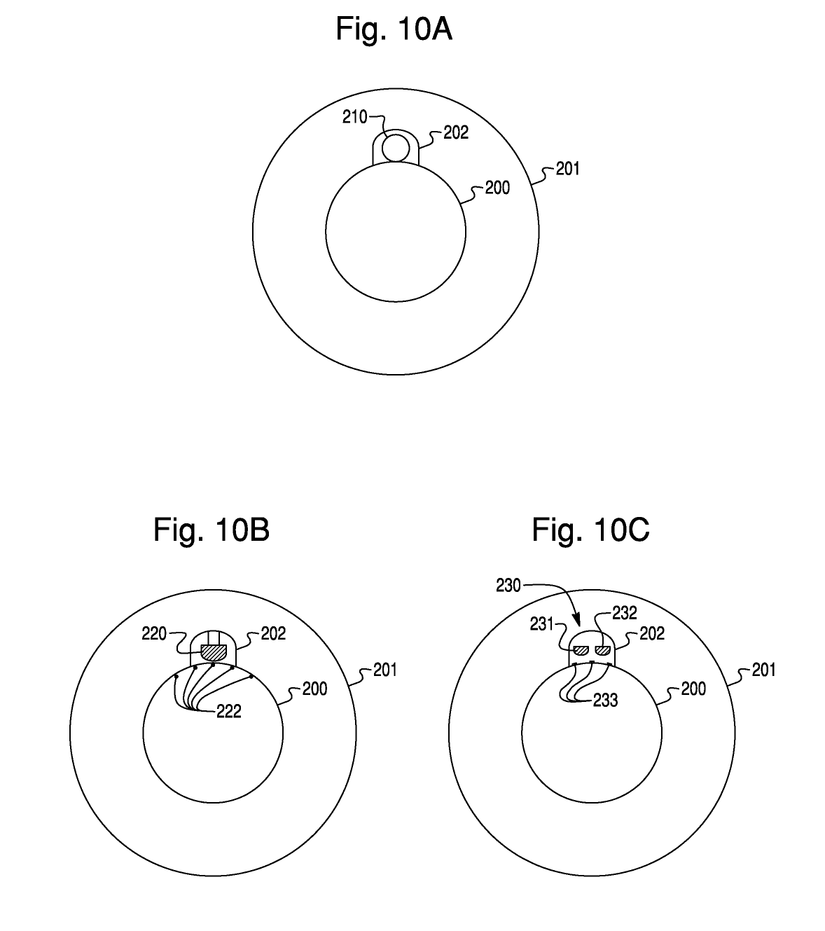

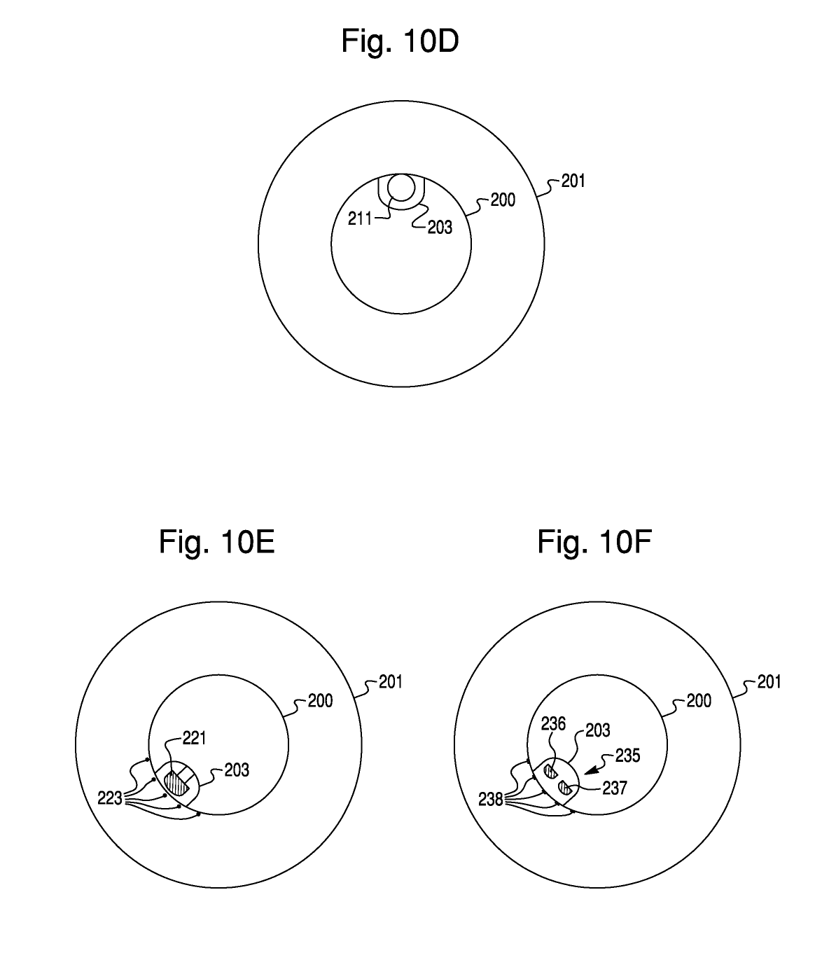

A number of rotational and/or translational motion sensors may be employed in this embodiment. Three example embodiments of such sensors are illustrated in FIGS. 10A, 10B and 10C. In these figures, a catheter 200 is shown surrounded by a sheath 201, which houses the sensor 210, 220, 230. Thus, the sensor 210, 220, 230 can sense rotational/translational motion with respect to the sheath 201. In procedures such as described above with respect to FIG. 4A, a sheath extending out of the patient from an artery guides the catheter to the location interest. In this configuration, the sheath does not move with respect to the patient, and therefore provides a reference for sensing catheter movement. Thus, the rotation/translation sensors can be built into the proximal end of the sheath 201. In other embodiments, a handle may be coupled to the proximal end of the sheath for guiding the catheter into the sheath, in which case the sensor 210, 220, 230 can be provided within the handle. In a further embodiment, catheter access to the vein may be by way of a trocar or other venal access port that is positioned within the patient, in which case the sensor 210, 220, 230 can be positioned within the trocar or femoral access port. In yet another embodiment, the sensor 210, 220, 230 can be positioned within a sleeve that can be held in place, such as taped or otherwise positioned on the patient, to provide a fixed reference point for measuring rotational/translational movements of the catheter. For expediency, each of these alternative embodiments is referred to commonly herein as a sheath 201, and the references to sheath 201 are not intended to exclude or preclude any of these alternative embodiments.

Referring to FIG. 10A, an embodiment of a rotational sensor includes a rotatable wheel 210 positioned within a volume 202 of the sheath 201 so as to be in contact with the catheter 200. When the catheter 200 is rotated within the sheath 201, the wheel 210 rotates. The rotational sensor senses catheter rotation by tracking turns of the wheel 210. Such a sensor may track wheel rotations by means of an axel coupled to the wheel 210 and an electronic rotation sensor, or by a magnetic or optical sensor (similar to the sensors described below with respect to FIGS. 10B and 10C) configured to sense wheel rotation. A rotational sensor employing a rotating wheel 210 in contact with the catheter 200 may be advantageous since it can be used with any catheter of sufficient outer diameter.

Referring to FIG. 10B, an embodiment of a rotational sensor includes a magnetic field sensor 220 within a volume 202 in the sleeve 201 which is configured to sense magnetic references 222 positioned at regular intervals about the catheter 200. The magnetic references 222 may be inlays or applied strips of ferromagnetic material which retains a magnetic field, examples of which include ferromagnetic material used in magstripe cards and floppy discs, and magnetic wires, such as iron or nickel. As the catheter 200 rotates within the sheath 201, magnetic references 222 move beneath the magnetic field sensor 220 which senses the movement as changes in the magnetic field. Rotation can be tracked by counting the direction and number of magnetic references 222 passing beneath the magnetic sensor 220. In a further embodiment, the magnetic references 222 include ferromagnetic materials capable of storing information in localized regions of magnetic field as well known in magstripe and magnetic tape technologies, and data is recorded in the magnetic references 222 sufficient to enable the magnetic field sensor 220 and connected processors to determine which individual reference lies beneath the sensor. For example, the magnetic references 222 may be narrow magnetic stripes each programmed with a different data corresponding to its position (e.g., angle) around the catheter 200. In this embodiment, for example, the magnetic field sensor 220 maybe similar to sensors used in computer hard drive storage devices, with the angle about the catheter 200 recorded in a few bits (e.g., 3 bits of data which requires a width of less than half a millimeter at the data density used in magnetic stripe cards). In this embodiment, the magnetic field sensor 220 can determine the angle of rotation of the catheter by reading the value from the magnetic reference 222 passing beneath it, thereby providing both a relative and absolute measure of rotation (i.e., change of magnetic field and angle information stored on the reference). In a further embodiment, the magnetic references 222 include a continuous stripe of magnetic material (e.g., magnetic tape) on which are recorded many zones of varying magnetic field which the magnetic field sensor 220 can detect.

Referring to FIG. 10C, an embodiment of a rotational sensor includes an optical sensor 230 within a volume 202 in the sleeve 201 which is configured to sense optical references 233 positioned at regular intervals about the catheter 200. The optical references 233 may be texture, markings or applied strips of reflective material. As the catheter 200 rotates within the sheath 201, optical references 233 move beneath the optical sensor 230 which senses the movement as changes in incident or reflected light. Rotation can be tracked by counting the direction and number of optical references 233 passing beneath the optical sensor 230. Such an optical sensor may be of any form well known in optical sensor technologies. For example, the optical sensor 230 may include a light source 231, such as a light emitting diode or tip of an optical fiber, positioned to shine light on the catheter 200 and optical references 233, and a light sensor 232, such as a photodiode, positioned to sense light reflected or emanating from the catheter 200. While the light source 231 and sensor 232 may be position within the volume 202 within the sheath 201, the source and sensor may be located elsewhere and optically linked to the volume 202 by one or more optical fibers. In this alternative configuration, a single optical fiber may be used to both transmit illuminating light to the volume 201 and return a portion of the reflected light. The optical sensor 230 may operate by sensing relative changes in reflected light, as would be produced when alternative bands of light and dark material pass beneath the sensor. In another embodiment, the optical sensor 230 may sense patterns (e.g., barcode) in the optical references 233, which may be used to encode information, such as angle about the catheter. In further embodiment, the optical sensor 230 may sense color so that angle information can be encoded by using different color optical references 233 about the catheter circumference.

While the sensors illustrated in FIGS. 10A-10C are shown as rotational sensors, these same sensors can be oriented to sense translational movement (i.e., into and out of the plane of the figures). That is, a rotation sensor 210 can include a sphere with the rotation sensor 210 configured to sense movement of the sphere in both rotational and translational directions. Similarly, magnetic references 222 may also be positioned circumferentially (e.g., a grid wrapped around the catheter 200) so that translational movements of the catheter 200 with respect to the sheath 201 can be sensed by the magnetic sensor 220. For example, the magnetic references 222 oriented circumferentially maybe of an opposite polarity than those oriented longitudinally so that the magnetic field sensor 220 can distinguish rotational from translational motion. Similarly, optical references 233 can be positioned circumferentially (e.g., a grid wrapped around the catheter 200) so that translational movements of the catheter 200 with respect to the sheath 201 can be sensed by the optical sensor 230. For example, the optical references 233 oriented circumferentially maybe of a different color or reflectivity (e.g., thinner or thicker) than those oriented longitudinally so that the optical sensor 230 can distinguish rotational from translational motion.

Alternative embodiments of rotational and/or translational movement sensors are illustrated in FIGS. 10D-10F wherein the sensor is positioned within the catheter and configured to sense rotation of the catheter with respect to surrounding sheath 201, handle, trocar or venal access port, or reference sleeve held in a fixed orientation with respect to the patient. Electrical leads from the catheter-based rotation/translation sensor for transmitting signals regarding rotational information to a processor can be included in the catheter electrical connector assembly. Since the proximal end of the catheter is not inserted into the patient, or inserted only a small distance within an introducer or sheath, the diameter of the proximal end of the catheter can be increased sufficient to accommodate the rotation and/or translational sensor. The embodiments illustrated in FIGS. 10D-10F permit the catheter to include all sensor electronics, reducing the cost of disposable sheaths 201, trocars or venal access ports.

Referring to FIG. 10C, an embodiment of a rotational sensor includes a rotatable wheel 211 positioned within a volume 203 of the catheter 200 so as to be in contact with the sheath 201. When the catheter 200 is rotated within the sheath 201, the wheel 211 rotates. The rotational sensor within the catheter senses catheter rotation by tracking turns of the wheel 211. Such a sensor may track wheel rotations by means of an axel coupled to the wheel 211 and an electronic rotation sensor, or by a magnetic or optical sensor (similar to the sensors described below with respect to FIGS. 10B and 10C) configured to sense wheel rotation. A rotational sensor employing a rotating wheel 211 in contact with the sheath 201 may be advantageous since it can be used with any sheath of appropriate inner diameter.

Referring to FIG. 10E, an embodiment of a rotational sensor includes a magnetic field sensor 221 within a volume 203 in the catheter 200 which is configured to sense magnetic references 223 positioned at regular intervals about the interior surface of the sheath 201. Similar to the embodiment illustrated in FIG. 10B, the magnetic references 223 may be inlays or applied strips of ferromagnetic material which retains a magnetic field, examples of which include ferromagnetic material used in magstripe cards and floppy discs, and magnetic wires, such as iron or nickel. As the catheter 200 rotates within the sheath 201, magnetic references 223 move over the magnetic field sensor 221 which senses the movement as changes in the magnetic field. Rotation can be tracked by counting the direction and number of magnetic references 223 passing over the magnetic sensor 221. In a further embodiment, the magnetic references 223 include ferromagnetic materials capable of storing information in localized regions of magnetic field as well known in magstripe and magnetic tape technologies, and data is recorded in the magnetic references 223 sufficient to enable the magnetic field sensor 221 and connected processors to determine which individual reference lies over the sensor. For example, the magnetic references 223 may be narrow magnetic stripes each programmed with a different data corresponding to its position (e.g., angle) around the sheath 201. In this embodiment, for example, the magnetic field sensor 221 maybe similar to sensors used in computer hard drive storage devices, with the angle about the sheath 201 recorded in a few bits (e.g., 3 bits of data which requires a width of less than half a millimeter at the data density used in magnetic stripe cards). In this embodiment, the magnetic field sensor 221 can determine the angle of rotation of the catheter by reading the value from the magnetic reference 223 passing over it, thereby providing both a relative and absolute measure of rotation (i.e., change of magnetic field and angle information stored on the reference). In a further embodiment, the magnetic references 223 include a continuous stripe of magnetic material (e.g., magnetic tape) on which are recorded many zones of varying magnetic field which the magnetic field sensor 221 can detect.

Referring to FIG. 10F, an embodiment of a rotational sensor includes an optical sensor 235 within a volume 203 in the catheter 200 which is configured to sense changes in optical characteristics of optical references 238 positioned at regular intervals about the interior surface of the sheath 201. The optical references 238 may be texture, markings or applied strips of reflective material. As the catheter 200 rotates within the sheath 201, optical references 238 move over the optical sensor 235 which senses the movement as changes in incident or reflected light. Rotation can be tracked by counting the direction and number of optical references 238 passing beneath the optical sensor 235. Such an optical sensor may be of any form well known in optical sensor technologies. For example, the optical sensor 235 may include a light source 236, such as a light emitting diode or tip of an optical fiber, positioned to shine light on the catheter 200 and optical references 238, and a light sensor 237, such as a photodiode, positioned to sense light reflected or emanating from the catheter 200. While the light source 236 and sensor 237 may be position within the volume 203 within the sheath 201, the source and sensor may be located elsewhere and optically linked to the volume 203 by one or more optical fibers. In this alternative configuration, a single optical fiber may be used to both transmit illuminating light to the volume 203 and return a portion of the reflected light. The optical sensor 235 may operate by sensing relative changes in reflected light, as would be produced when alternative bands of light and dark material pass above the sensor. In another embodiment, the optical sensor 235 may sense patterns (e.g., barcode) in the optical references 238, which may be used to encode information, such as angle about the catheter. In further embodiment, the optical sensor 235 may sense color so that angle information can be encoded by using different color optical references 238 about the catheter circumference.

Information from a rotational and/or translational movement sensor at the catheter proximal end can be provided to a sensor, such as the ultrasound imaging system processor, which can be programmed to interpret the sensor information and estimate the position and orientation of the catheter distal end based upon that information. This processor may estimate the catheter position in real time, or store the sensor information along with ultrasound image information for later processing. Also, the processor may be programmed to update position and orientation information obtained from fluoroscopic methods with rotational and/or translational movement sensor information.

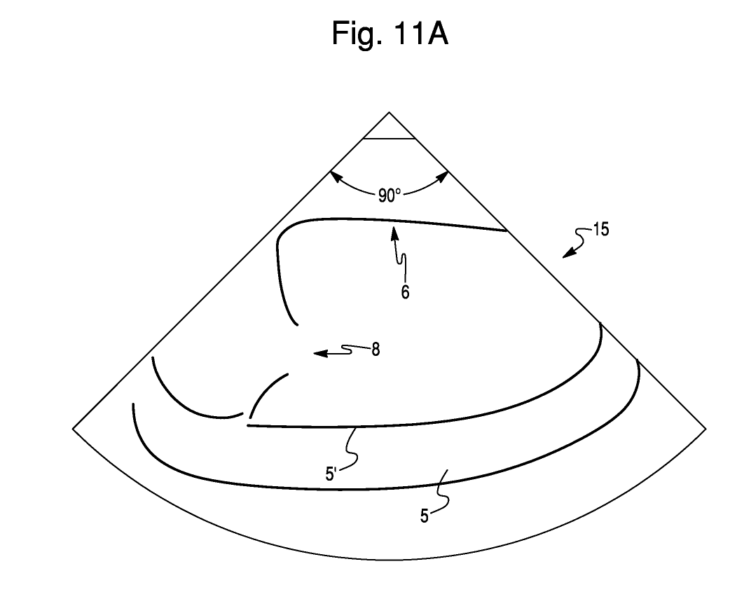

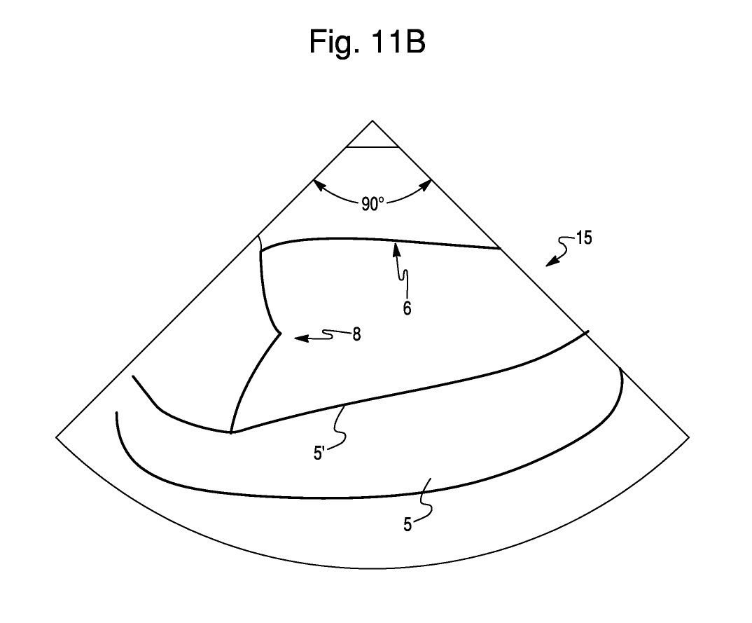

An ultrasound phased array transducer operated using B-mode ultrasound imaging technique renders 2D images, such as the images of the left ventricle of the heart illustrated in FIGS. 11A and 11B. B-Mode ultrasound imaging displays an image representative of the relative echo strength received at the transducer. A 2-D image can be formed by processing and displaying the pulse-echo data acquired for each individual scan line across the angle of regard 15 of the phased array transducer. This process yields a two-dimensional B-mode image of the endocardial surface of the ventricle, examples of which is illustrated in FIGS. 11A and 11B. The cardiologist may define the edge of the endocardial surface 5′, 7′ in the image by manually tracing the edge using an interactive cursor (such as a trackball, light pen, mouse, or the like) as may be provided by the ultrasound imaging system. By identifying the edges of structure within an ultrasound image, an accurate outline of ventricle walls can be obtained and other image data ignored. The result of this analysis may be a set of images and dimensional measurements defining the position of the ventricle walls at the particular instants within the cardiac cycle. The dimensional measurements defining the interior surface 5′ or 7′ of the endocardium can be stored in memory of the ultrasound system and analyzed using geometric algorithms to determine the volume of the ventricle.

The system computer can then combine the position information with the ECG data obtained through any of the means currently known in the art, and the 2D images acquired from the ultrasound scanner (100). The ECG signals, which generally correlate to the phase of the heart in the cardiac cycle, can be used to judge whether frames acquired over a number of cardiac cycles correspond to relatively similar mechanical states of contraction or relaxation from one acquired frame to the next acquired frame. Methods for using ECG signals to combine images and average multiple images at a particular phase or relative time within the cardiac cycle (time gating) are described in U.S. application Ser. No. 11/002,661 published as U.S. Patent Publication No.2005/0080336, the entire contents of which are incorporated herein by reference in their entirety.

Correlating ultrasound images to phases in the cardiac cycle can be important since otherwise, different parts of the reconstructed 3D image might be obtained from different parts of the cardiac cycle and provide an inaccurate representation of the cardiac structure being imaged. However, care has to be taken to ensure that there exists no appreciable time-difference between the ECG signals and the ultrasonic images, since such delays could be entrained when dealing with large amounts of image data as compared to the relatively low density of data from a few ECG channels.

Additional system components may be provided as would be readily apparent to one of ordinary skill in the art after reading this disclosure.

An embodiment of a method of processing 2D ultrasound images according to an embodiment of the present invention is illustrated in the flowchart of FIG. 12. It should be appreciated that, as with other methods described herein, the method shown in FIG. 12 may be implemented using the exemplary imaging system of FIG. 1, or using another suitable imaging system.

Referring to FIG. 12, in step 900 the imaging system obtains the ECG of the subject under investigation. The relative phase of the cardiac cycle is judged from the ECG and, if appropriate (905), as described in this disclosure, the corresponding image along with the position data of the ultrasound transducer is obtained (925, 910, 915, and 920). The ultrasound image referred to herein can include image data in various formats. The data can also originate from one or more parts of the ultrasound image processing chain, including but not limited to RF data, pre scan conversion data, or scan-converted digital data, etc., as would be apparent to one of ordinary skill in the art from reading this disclosure. The above technique can also include continuous collection of ECG, image, and position data followed by later selection of useable data, based on the cardiac cycle, from such a collection during post processing.

The acquired data can then be arranged dynamically in a 3-D data matrix with appropriate recalculation based on positional information (930). Any number of algorithms may be used for accomplishing this. Generally, the image data will be stored in memory. Time and ECG phase information can be stored with the images or correlated to the images so that each image can be matched to a particular time in the cardiac cycle. Additionally, the position information determined through fluoroscopy associated with each image can be stored in memory, either with the image or so that it can be matched to the images. The sensor position information may be stored as a series of three dimensional vectors (i.e., the instantaneous position information) determined by the fluoroscopic technique described herein for each movement of the catheter. The resulting data set may be a data table of images, ECG base information, and corresponding sensor positional information (i.e., the determined fluoroscopic position image at the time each image is obtained by the imaging transducer). Alternatively, the images, ECG. and positional information may be stored as three separate data tables that are linked by means of an index or a pointer array. Further image processing, such as to enhance image quality or representation, and reduce noise, might also be applied prior to such 3-D voxel determination.

With the image, ECG and position information stored in memory, the processor can correlate, combine or average images of a particular point in the cardiac cycle. This may be accomplished by reorganizing the data table, or, more likely, including metadata which allows the processor to quickly identify images at common points in the cardiac cycle.

When all required frames are collected (steps 940, 935; with the former being defined directly or indirectly by the user), a 3-D image can be generated by “stitching” together adjoining images using methods similar to how digital photographs can be stitched together to create panorama images.

Once a 3-D or 4-D dataset has been generated by the processor, the information can be represented on any appropriate user interface in step 955. This may be accomplished by any number of display algorithms. For example, the processor can map the 3-D information into 2-D display by means of raster graphics techniques. Alternatively, the data may be mapped to a model of the heart in order to better indicate the sensed structure in a form that the physician can interpret. Such user interface could also include post processing apparent to one of ordinary skill in the art, such as rotation, lighting, sectioning and such other common tools of 3-D data manipulation and representation well known in the computer graphics arts.

A processor coupled to linear and rotational sensor such as described above can be configured to continuously monitor either position or a change in rotation as well as, optionally, translation of the catheter shaft with respect to the sheath 201. Whenever a change in position of the proximal end of the catheter is detected, the processor can update the relative orientation and position of the catheter distal end (and thus the transducer array) that is correlated with ultrasound images. This updated position/rotation information can then be associated with the ultrasound image obtained until a further rotation or translation is sensed. Periodically, the true position and rotational orientation of the transducer array may be determined using the fluoroscopic methods described above, at which point the processor may then update the position/orientation information that is correlated with ultrasound images, as well as update calibration adjustments applied by the processor to catheter proximal end rotational/translational movement measurements in order to estimate changes of position of the distal end of the catheter.

Alternatively or in addition to the methods described above, the calculation of position can be simplified by using a catheter that can be held in predictable positions. Provided by either designing a catheter that has a high flexural stiffness through rotation spanning at least the volume of interest, or by limiting the flexural movement of the imaging array by encapsulating it within a mechanically limiting outer sheath with an acoustical window through which a clinically acceptable image can be obtained. In this manner, the processor need not account for the flexure of that catheter, or the flexure can be calculated as the difference between the angle measured by the methods described above and the angle indicated by the rotation of the catheter at its base (i.e., proximal end). Thus, a measure of rotation obtained at the base of the catheter and can be extrapolated to determine the positional orientation of the tip of the catheter several inches away. According to one embodiment, the imaging catheter is introduced through a long sheath with favorable acoustic properties.

In conjunction with the methods described herein, a number of other techniques may be used for detecting baseline and changes to the position and orientation of the catheter within the heart at a particular instance. Disclosure of methods of measuring movements of an ultrasound imaging catheter using accelerometers is provided in U.S. Patent Application TBD, entitled “Catheter Position Tracking for Intracardiac Catheters”, filed concurrently with the present application and incorporated herein by reference in its entirety. Other methods of locating the catheter employ the use of sound (i.e., echo location) or magnetic fields to measure the position using triangulation methods. Examples of suitable echo location position sensors are disclosed in U.S. application Ser. No. 10/994,424, previously incorporated by reference. As described earlier, such tracking need not necessarily include rotational position. In such instances, a combination of the rotational position tracking disclosed herein (e.g., the X-ray imaging techniques described above) along with the position tracking of the acoustic array by magnetic, acoustic, or electrical means can be combined to obtain the necessary data to enable the processor to assemble 2-D image slices into 3D images according to the various embodiments.

The frequency of baseline position measurements, accelerometer provided displacement measurements, rotational position estimations using any of the techniques previously described, and the imaging frame rate may need to be sufficiently high to provide the degree of resolution required by the particular diagnostic objective for the examination. Further, the positional and rotational measurements and ultrasound imaging may need to be timed such that all three of these measurements/estimations are within an acceptable time-span or time-correlation error band to permit clinically acceptable 3-D representation. This latter concern may arise because the duration required to record each measurement or image may be different and there will be an error (i.e., degree of uncertainty) associated with the time at which each ultrasound image is obtained. If such errors are not properly managed or otherwise taken into account, the result may be a blurring of the generated 3-D images.

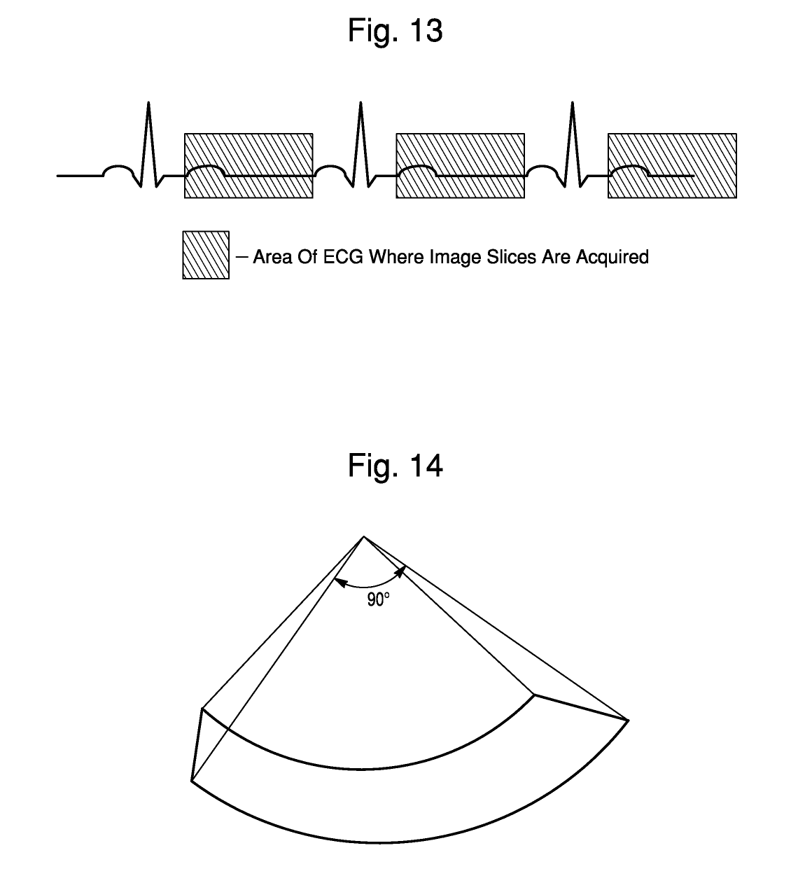

FIG. 13 shows a representation of the human electrocardiogram (ECG). Highlighted sections show areas of ventricle diastole, where the cardiac muscle activity is minimal. It is at the end of this phase of the cardiac cycle that the ventricles of the heart are at their maximum volume. More precisely, during Diastole the ventricles relax as the ventricle muscles repolarize (evidenced by the T wave) and enlarge as the atria are emptying into the ventricles. The P wave corresponds to the depolarization of the atria by the Sinoatrial (SA) node, which causes a last squeeze in the atria to push the remaining blood into the ventricle. This is called atrial systole. Thus, the ECG signal provides important timing information related to the shape and motion of the heart chambers. Images acquired during the ventricle diastole phase of the cardiac cycle are useful for reconstructing 3-D images of the heart, since a maximum number of ultrasound images can be acquired of the ventricle in the relatively longer time-spans during which the heart assumes a particular shape. In cases where abnormal conditions exist, such as rhythm abnormalities, and where such abnormality is atrial fibrillation in particular, such periods of mechanical inactivity might be too short or even absent. In such situations, multiple frames may need to be acquired and averaged to reduce the overall spatial error in the estimation of the 3-D image.

By limiting ultrasound imaging to the time between heart beats, from the end of the repolarization of the previous beat to the start of the next depolarization, as illustrated in FIG. 13, distortions and artifacts in the catheter location estimates caused by muscle movement may be avoided. By reducing distortions and artifacts in catheter position caused by muscle movement, sequences of ultrasound images can be more accurately combined to generate 3-D images of the heart. If the heart is assumed to be relatively still (i.e., static), then overlapping images can be easily stitched together to generate a 3-D image.

In an embodiment, images can be acquired along a given plane throughout a cardiac cycle. Multiple, spatially adjacent planes correlated temporally can then yield 3-D representations of the mechanical activity of the heart.

FIG. 14 shows an isometric projection of the acquired image volume, assuming that the underlying imaging technique is a phased scan. However, other scans such as linear scans and circular scan profiles can also be employed in alternative embodiments.

The aforementioned embodiments assume that the underlying imaging is carried out through what is generally known in the industry as B-mode imaging. Additional embodiments include 3-D reconstruction of Color Doppler data, with data separation between underlying tissue and Color Flow information and with the possibility of data separation between different flow directions. Such embodiments use the same methods as the embodiments described above for locating the instantaneous position of the imaging sensor except that the image data is obtain in M-Mode or Color Doppler mode.

The foregoing embodiments enable a 3-D reconstruction of ultrasound images of the heart. Such embodiments may be particularly useful when the patient is in Atrial Fibrillation or flutter. In such situations, the heart is flexing in irregular and unpredictable patterns that may be disjoint from the ECG patterns. In such conditions, methods that use ECG signals to assist in forming a 3-D image may be infeasible. This may be true especially in conditions of Atrial Fibrillation when the motions of the atrial walls are random, although small.

In yet another embodiment, the 3-D and 4-D image data sets generated according to various embodiments are combined with image data from one or more external sources. For example, fluoroscopic images of the heart (including images used to determine transducer array position and orientation) may be correlated to absolute time or ECG data and thereby correlated to particular 3-D ultrasound image datasets. Such correlated images then can be presented as overlapping or otherwise merged images on a display. This embodiment may enable physicians to see structures outside of the ultrasound image scan (e.g., behind the transducer array or beyond the imaging range of the transducer) as they match up with ultrasound imaged structures.

Imaging the heart in 3-D and 4-D according to the various embodiments can be used to aid a physician in identifying locations for position a pacing lead on the heart in order to provide optimum benefit from a pacemaker. By generating accurate images of the surface of the heart throughout the cardiac cycle, the physician can locate regions that are not contracting in sequence or to the full extent as adjoining regions. By positioning pacing leads on such regions of the heart, a pacemaker may be better able to provide pacing stimulation to regions of the heart where such stimulation is most beneficial. Additionally, by imaging the heart in four dimensions, the physician can identify regions (such as portions of the left or right ventricle or atria) that are contracting early, late or otherwise out of phase with the rest of the heart. Lagging regions may be appropriate for placement of pacing leads. Additionally, the information on contraction lag contained in such a 4-D image dataset can be used to set the pace maker timing parameters. In this embodiment, the physician uses the 4-D image dataset (such as by running the 3-D images forward and backwards in time on the display) to calculate the time at which a lagging region of the heart should have contracted and uses this calculation to set the pacemaker timing parameter.

While the present invention has been disclosed with reference to certain exemplary embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

1. An ultrasound imaging system, comprising:
a catheter including:
an ultrasound imaging transducer array disposed near a distal end of the catheter, and
a connecting harness disposed proximate to the imaging transducer array;
a fluoroscope imager configured to obtain an X-ray image of the ultrasound imaging transducer array and connecting harness when the catheter is positioned within a patient; and
a processor configured to receive data from both the fluoroscope imager and adapted to determine a rotational orientation of the ultrasound imaging transducer based upon the X-ray image of the ultrasound imaging transducer array and connecting harness.

2. The ultrasound imaging system as in claim 1, wherein the processor is further configured to determine the ultrasound imaging transducer array position within the patient based upon the X-ray image of the imaging transducer array.

3. The ultrasound imaging system as in claim 1, wherein the processor is further configured to receive ultrasound image information from the catheter and generate a three-dimensional ultrasound image based upon the ultrasound image information and the determined rotational orientation.

4. The ultrasound imaging system as in claim 1, wherein the processor is further adapted to determine the rotational position of the imaging transducer array based upon dimensions and extent of overlap of the images of the imaging transducer array and the connecting harness.

5. The ultrasound imaging system as in claim 1, wherein the catheter further includes a radio-opaque material positioned in the catheter near the ultrasound imaging transducer array and oriented to have an axis of symmetry different from that of the ultrasound imaging transducer array.

6. The ultrasound imaging system as in claim 1, further comprising a rotational sensor configured to sense rotation of a proximal end of the catheter and provide information regarding the sensed rotation to the processor,
wherein the processor is further adapted to estimate the rotational orientation of the imaging transducer array based upon the sensed rotation information.

7. The ultrasound imaging system as in claim 6, further comprising a translational sensor configured to sense translational motion of the proximal end of the catheter and provide information regarding the sensed translational motion to the processor,
wherein the processor is further adapted to estimate the position of the imaging transducer array based upon the sensed translational motion information.

8. A method of determining an orientation of an ultrasound imaging transducer array positioned at a distal end of an ultrasound imaging catheter positioned within a patient, comprising:
imaging the ultrasound imaging transducer array using fluoroscopy;
measuring dimensions of a fluoroscopic image of the ultrasound imaging transducer array and a connecting harness; and
determining the orientation of the ultrasound imaging transducer array based upon the measured dimensions.

9. The method of determining an orientation of an ultrasound imaging transducer array of claim 8, wherein the ultrasound imaging catheter includes a radio-opaque material positioned in the catheter near the ultrasound imaging transducer array and oriented to have an axis of symmetry different from that of the ultrasound imaging transducer array; and further comprising:
measuring dimensions of the radio-opaque material; and
determining the orientation of the ultrasound imaging transducer array based also upon the measured the radio-opaque material dimensions.

10. The method of determining an orientation of an ultrasound imaging transducer array of claim 8, further comprising providing fluoroscopic images of the ultrasound imaging transducer to a processor,
wherein the processor performs the steps of measuring dimensions and determining the orientation of the ultrasound imaging transducer array.

11. The method of determining an orientation of an ultrasound imaging transducer array of claim 10, further comprising:
sensing a rotation of a proximal end of the catheter; and
providing information regarding the sensed rotation to the processor,
wherein determining the orientation of the ultrasound imaging transducer array is also based upon the information regarding the sensed rotation.

12. The method of determining an orientation of an ultrasound imaging transducer array of claim 11, further comprising:
sensing a translational motion of the proximal end of the catheter;
providing information regarding the sensed translational motion to the processor; and
determining a position of the ultrasound imaging transducer array based upon fluoroscopic images of the ultrasound imaging transducer and the information regarding the sensed translational motion.

13. An ultrasound imaging catheter, comprising a rotational sensor configured to sense rotation of a proximal end of the catheter.

14. The ultrasound imaging catheter of claim 13, further comprising:
a translational motion sensor configured to sense translational motion of the proximal end of the catheter.

15. The ultrasound imaging catheter of claim 13, wherein the rotational sensor senses rotation of the catheter with respect to a sheath.

16. The ultrasound imaging catheter of claim 14, wherein the translational motion sensor senses translational motion of the catheter with respect to a sheath.

17. The ultrasound imaging catheter of claim 15, wherein the rotational sensor comprises a roller positioned within the catheter so as to contact an inner surface of the sheath.

18. The ultrasound imaging catheter of claim 15, wherein the rotational sensor comprises a magnetic field sensor positioned within the catheter and configured to sense magnetic fields within an interior of the sheath.

19. The ultrasound imaging catheter of claim 15, wherein the rotational sensor comprises an optical sensor positioned within the catheter to sense changes in optical characteristics of an inner surface of the sheath.

20. The ultrasound imaging catheter of claim 19, wherein the wherein the optical sensor includes a light emitting diode and a photodiode.

21. The ultrasound imaging catheter of claim 13, further comprising:
an ultrasound imaging transducer array; and
a radio-opaque material positioned in the catheter near the ultrasound imaging transducer array and oriented to have an axis of symmetry different from that of the ultrasound imaging transducer array.

22. The ultrasound imaging catheter of claim 14, further comprising:
an ultrasound imaging transducer array; and
a radio-opaque material positioned in the catheter near the ultrasound imaging transducer array and oriented to have an axis of symmetry different from that of the ultrasound imaging transducer array.

23. The method of determining an orientation of an ultrasound imaging transducer array of claim 11, further comprising:
sensing a translational motion of the proximal end of the catheter;

24. A method of determining an orientation of an ultrasound imaging transducer array positioned at a distal end of an ultrasound imaging catheter positioned within a patient, comprising:
measuring a rotation of the catheter using a rotation sensor positioned near a proximal end of the catheter;
communicating rotation measurement information from the rotation sensor to a processor; and
estimating the orientation of the ultrasound imaging transducer array based upon the rotation measurement information, wherein the orientation estimating is accomplished by the processor.

25. The method of determining an orientation of an ultrasound imaging transducer array of claim 24, further comprising:
sensing a translational motion of the catheter using a translation motion sensor positioned near a proximal end of the catheter;
communicating translation motion measurement information from the translation motion sensor to the processor; and
estimating the ultrasound imaging transducer array position within the patient based upon the measured translational motion of the catheter, wherein the position estimating is accomplished by the processor.

26. An ultrasound imaging catheter, comprising:
an ultrasound imaging transducer array having an axis of symmetry; and
a radio-opaque material positioned in the catheter near the ultrasound imaging transducer array and oriented to have an axis of symmetry at an acute angle to the axis of symmetry of the ultrasound imaging transducer array.

27. A method of generating a three-dimensional ultrasound image of an organ, comprising:
deploying a catheter within the organ, the catheter having an ultrasound imaging transducer array disposed near a distal end of the catheter;
obtaining an X-ray image of the organ and catheter;
determining a baseline linear position of the ultrasound imaging transducer array from the X-ray image;
determining a baseline rotational orientation of the ultrasound imaging transducer array by measuring dimensions of the ultrasound imaging transducer array and connecting harness in the X-ray image;
obtaining an ultrasound image using the ultrasound imaging transducer array;
manipulating the catheter to change the orientation of the ultrasound imaging transducer array;
repeating the steps of determining the linear position and rotational orientation of the ultrasound imaging transducer array and obtaining an ultrasound image; and
generating the three-dimensional image by combining the obtained ultrasound images based upon the determined positions and orientations of the ultrasound imaging transducer array corresponding to each ultrasound image.

28. The method of generating a three-dimensional ultrasound image of an organ of claim 27, wherein the organ is a heart, and further comprising obtaining ultrasound images throughout a cardiac cycle and generating three-dimensional images of the heart throughout the cardiac cycle.

29. The method of generating a three-dimensional ultrasound image of an organ of claim 28, further comprising locating a position for attaching a pacemaker pacing lead based upon the three-dimensional images of the heart throughout the cardiac cycle.
